(19) United States
(12) Patent Application Publication
(10) Pub. No.: US 2019/0269380 A1
(43) Pub. Date: Sep. 5, 2019

(54) BLOOD FLOW DISPLAYING METHOD, APPARATUS AND DEVICE FOR ULTRASONIC SYSTEM

(71) Applicant: Edan Instruments, Inc, Shenzhen (CN)

(72) Inventors: Fengjiao Li, Shenzhen (CN); Seshadri Srinivasan, Sunnyvale, CA (US)

(21) Appl. No.: 16/135,575

(22) Filed: Sep. 19, 2018

(30) Foreign Application Priority Data
Mar. 5, 2018 (CN) 201810179560.6

(51) Int. Cl. A61B 8/06; A61B 8/00

(52) U.S. Cl. CPC A61B 8/06; A61B 8/5246; A61B 8/463

(57) ABSTRACT

Provided are a blood flow displaying method, apparatus and device for an ultrasonic system. The method includes: determining a current pulse recurrence frequency of the ultrasonic system, determining a current extraction factor according to the current pulse recurrence frequency and a preset reference pulse recurrence frequency, extracting a target gradation sequence from a preset gradation set according to the current extraction factor, and displaying a blood flow velocity currently detected by the ultrasonic system using a spectrum of the target gradation sequence.

### CROSS-REFERENCE TO RELATED APPLICATION

This application claims priority to and benefits of Chinese Patent Application Serial No. 201810179560.6, filed with the State Intellectual Property Office of P. R. China on Mar. 5, 2018, the entire content of which is incorporated herein by reference.

### FIELD

The present disclosure relates to the field of medical instruments, and more particularly to a blood flow displaying method, apparatus and device for an ultrasonic system.

### BACKGROUND

In clinical applications, in order to reflect true situations of human tissues and organs more completely and effectively, a color flow (CF) mode is usually superimposed with a brightness (B) mode of an ultrasonic equipment, such that pulses are transmitted repeatedly to a detected site, blood flow velocities are obtained according to a correlation between pulses, and are displayed in different colors and depths so as to real-timely obtain color flow images, based on which, a doctor may observe blood flow characteristics so as to discover a disease timely.

At present, for the flow image display of the ultrasonic equipment in the CF mode, it is common that blood flow velocities or motion velocities of other tissues are calculated and directly mapped to gradations of a fixed spectrum, so as to display different blood flow velocities in different colors and depths. In this way, however, a dynamic range of the blood flow varies very little under high pulse recurrence frequency (prf), such that color display of the blood flow is mostly concentrated at a lower part of the spectrum in poor gradation, based on which medical staffs cannot well distinguish a low flow velocity from a high flow velocity, so as to further affect the accuracy and reliability of the diagnosis. Moreover, the higher the pulse recurrence frequency is, the more serious the above defects are, thereby seriously affecting the beauty of the flow image display and diagnosis results.

### SUMMARY

Embodiments of the present disclosure seek to solve at least one of the problems existing in the related art to at least some extent.

According to a first aspect of embodiments of the present disclosure, a blood flow displaying method for an ultrasonic system is provided, the method includes: determining a current pulse recurrence frequency of the ultrasonic system; determining a current extraction factor according to the current pulse recurrence frequency and a preset reference pulse recurrence frequency; extracting a target gradation sequence from a preset gradation set according to the current extraction factor; and displaying a blood flow velocity currently detected by the ultrasonic system using a spectrum of the target gradation sequence.

According to a second aspect of embodiments of the present disclosure, there is provided a computer device, including: a processor; and a memory having stored therein computer programs executable by the processor, when executed by the processor, the computer programs cause the processor to implement the blood flow displaying method for an ultrasonic system as described in the first aspect of embodiments of the present disclosure.

According to a third aspect of embodiments of the present disclosure, there is provided a computer-readable storage medium having stored therein computer programs that, when executed by a processor, causes the processor to implement the blood flow displaying method for an ultrasonic system as described in the first aspect of embodiments of the present disclosure.

Additional aspects and advantages of embodiments of present disclosure will be given in part in the following descriptions, become apparent in part from the following descriptions, or be learned from the practice of the embodiments of the present disclosure.

### BRIEF DESCRIPTION OF THE DRAWINGS

These and other aspects and advantages of embodiments of the present disclosure will become apparent and more readily appreciated from the following descriptions made with reference to the drawings, in which:

FIG. 1 is a flow chart of a blood flow displaying method for an ultrasonic system according to embodiments of the present disclosure;

FIG. 2 is a schematic diagram of a preset gradation set having 1024 gradations according to embodiments of the present disclosure;

FIG. 3 is a flow chart of a blood flow displaying method for an ultrasonic system according to embodiments of the present disclosure;

FIG. 4 is a schematic diagram of a gradation sequence corresponding to a preset reference pulse recurrence frequency according to embodiments of the present disclosure;

FIG. 5 is a schematic diagram showing a display of blood flow velocity information using a gradation sequence corresponding to a preset reference pulse recurrence frequency according to embodiments of the present disclosure;

FIG. 6 is a schematic block diagram of a blood flow displaying apparatus for an ultrasonic system according to embodiments of the present disclosure;

FIG. 7 is a schematic diagram of a computer device according to embodiments of the present disclosure;

FIG. 8 is a schematic diagram of a blood flow displaying device according to embodiments of the present disclosure; and

FIG. 9 illustrates comparisons between blood flow displaying results obtained by a blood flow displaying method according to embodiments of the present disclosure and that obtained by a method in related art.

### DETAILED DESCRIPTION

Reference will be made in detail to embodiments of the present disclosure. The embodiments described herein with reference to drawings are explanatory, illustrative, and used to generally understand the present disclosure. The embodiments shall not be construed to limit the present disclosure. The same or similar elements and the elements having same or similar functions are denoted by like reference numerals throughout the descriptions.

In the related art, a dynamic range of the blood flow varies very little under high pulse recurrence frequency (prf), such that when blood flow velocities or motion velocities of other tissues are calculated and mapped to gradations, color display of the blood flow is mostly concentrated at a lower part of a spectrum in poor gradation, based on which medical staffs cannot well distinguish a low flow velocity from a high flow velocity, so as to further affect the accuracy and reliability of the diagnosis. Moreover, the higher the pulse recurrence frequency is, the more serious the above defects are, thereby seriously affecting the beauty of the flow image display and diagnosis results. For these problems, embodiments of the present disclosure provide a blood flow displaying method, apparatus and a device for an ultrasonic system.

According to a first aspect of embodiments of the present disclosure, a blood flow displaying method for an ultrasonic system is provided, the method includes: determining a current pulse recurrence frequency of the ultrasonic system; determining a current extraction factor according to the current pulse recurrence frequency and a preset reference pulse recurrence frequency; extracting a target gradation sequence from a preset gradation set according to the current extraction factor; and displaying a blood flow velocity currently detected by the ultrasonic system using a spectrum of the target gradation sequence.

With the blood flow displaying method for an ultrasonic system provided in embodiments of the present disclosure, by determining the current pulse recurrence frequency of the ultrasonic system, determining the current extraction factor according to the current pulse recurrence frequency and the preset reference pulse recurrence frequency, extracting the target gradation sequence from the preset gradation set according to the current extraction factor, and displaying the blood flow velocity currently detected by the ultrasonic system using the spectrum of the target gradation sequence, the target gradation sequence is obtained, and the blood flow is displayed using the spectrum of the target gradation sequence. Therefore, the blood flow display is not only enhanced in gradation, but also may be optimized according to requirements of medical staffs, which greatly improves the flexibility of the blood flow display, and provides reliable basis and conditions for accurate diagnosis and analysis of the medical staffs.

Alternatively, in an embodiment of the present disclosure, determining a current extraction factor includes determining the current extraction factor according to a ratio of the current pulse recurrence frequency to the preset reference pulse recurrence frequency.

Alternatively, in an embodiment of the present disclosure, extracting a target gradation sequence from a preset gradation set includes: extracting the target gradation sequence from the preset gradation set according to the current extraction factor if the current extraction factor is greater than or equal to 1; or determining a gradation sequence corresponding to the preset reference pulse recurrence frequency as the target gradation sequence if the current extraction factor is less than 1.

Alternatively, in an embodiment of the present disclosure, the preset gradation set includes M gradations, and the number of gradations displayed by the ultrasonic system is N, and N is less than M; before determining the gradation sequence corresponding to the preset reference pulse recurrence frequency as the target gradation sequence, the method further includes: extracting N gradations from the M gradations according to a preset rule to constitute the gradation sequence corresponding to the preset reference pulse recurrence frequency.

Alternatively, in an embodiment of the present disclosure, extracting N gradations from the M gradations according to a preset rule includes: extracting N/2 gradations starting from a central position to each of two ends of the M gradations, respectively.

Alternatively, in an embodiment of the present disclosure, if the current extraction factor k is a non-integer greater than 1, and k=a/b where both a and b are positive integers, extracting the target gradation sequence from the preset gradation set according to the current extraction factor includes: determining the target gradation sequence through an interpolation processing according to the current extraction factor and gradations included in the preset gradation set; or extracting b gradations per a gradations of the preset gradation set to constitute the target gradation sequence.

Alternatively, in an embodiment of the present disclosure, displaying a blood flow velocity currently detected by the ultrasonic system using a spectrum of the target gradation sequence includes: determining a blood flow velocity range corresponding to the current pulse recurrence frequency; and one-to-one mapping the blood flow velocity range to the spectrum of the target gradation sequence successively.

According to a second aspect of embodiments of the present disclosure, there is provided a computer device, including: a processor; and a memory having stored therein computer programs executable by the processor, when executed by the processor, the computer programs cause the processor to implement the blood flow displaying method for an ultrasonic system as described in the first aspect of embodiments of the present disclosure.

With the computer device provided in embodiments of the present disclosure, by determining the current pulse recurrence frequency of the ultrasonic system, determining the current extraction factor according to the current pulse recurrence frequency and the preset reference pulse recurrence frequency, extracting the target gradation sequence from the preset gradation set according to the current extraction factor, and displaying the blood flow velocity currently detected by the ultrasonic system using the spectrum of the target gradation sequence, the target gradation sequence is obtained, and the blood flow is displayed using the spectrum of the target gradation sequence. Therefore, the blood flow display is not only enhanced in gradation, but also may be optimized according to requirements of medical staffs, which greatly improves the flexibility of the blood flow display, and provides reliable basis and conditions for accurate diagnosis and analysis of the medical staffs.

According to a third aspect of embodiments of the present disclosure, there is provided a computer-readable storage medium having stored therein computer programs that, when executed by a processor, causes the processor to implement the blood flow displaying method for an ultrasonic system as described in the first aspect of embodiments of the present disclosure.

According to a fourth aspect of embodiments of the present disclosure, a blood flow displaying apparatus for an ultrasonic system is provided, the apparatus includes: a first determining module configured to determine a current pulse recurrence frequency of the ultrasonic system; a second determining module configured to determine a current extraction factor according to the current pulse recurrence frequency and a preset reference pulse recurrence frequency; an extracting module configured to extract a target gradation sequence from a preset gradation set according to the current extraction factor; and a displaying module configured to display a blood flow velocity currently detected by the ultrasonic system using a spectrum of the target gradation sequence.

With the blood flow displaying apparatus for an ultrasonic system provided in embodiments of the present disclosure, by determining the current pulse recurrence frequency of the ultrasonic system, determining the current extraction factor according to the current pulse recurrence frequency and the preset reference pulse recurrence frequency, extracting the target gradation sequence from the preset gradation set according to the current extraction factor, and displaying the blood flow velocity currently detected by the ultrasonic system using the spectrum of the target gradation sequence, the target gradation sequence is obtained, and the blood flow is displayed using the spectrum of the target gradation sequence. Therefore, the blood flow display is not only enhanced in gradation, but also may be optimized according to requirements of medical staffs, which greatly improves the flexibility of the blood flow display, and provides reliable basis and conditions for accurate diagnosis and analysis of the medical staffs.

In the following, a blood flow displaying method, apparatus and device for an ultrasonic system according to embodiments of the present disclosure will be described in detail referring to drawings.

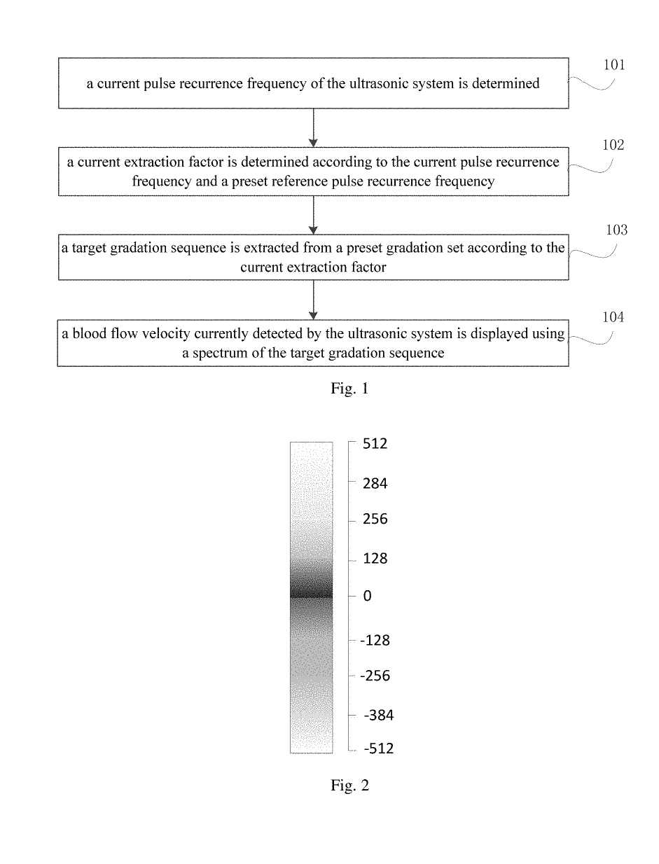

FIG. 1 is a flow chart of a blood flow displaying method for an ultrasonic system according to embodiments of the present disclosure.

With reference to FIG. 1, the blood flow displaying method for an ultrasonic system according to embodiments of the present disclosure will be illustrated in detail below.

As shown in FIG. 1, the blood flow displaying method for an ultrasonic system according to embodiments of the present disclosure may include the following operations at blocks illustrated in FIG. 1.

At block 101, a current pulse recurrence frequency of the ultrasonic system is determined.

Specifically, the blood flow displaying method for an ultrasonic system in embodiments of the present disclosure may be carried out by the blood flow displaying apparatus for an ultrasonic system of the present disclosure, and the apparatus is configured in a computer device of the present disclosure to control the blood flow display.

The computer device in embodiments of the present disclosure may be any hardware device having the ultrasonic system, which will not be specifically limited in present disclosure.

In an implementation, the current pulse recurrence frequency of the ultrasonic system may be determined by querying a function setting module of the blood flow displaying apparatus for an ultrasonic system or by other means, which will not be specifically limited in present disclosure.

At block 102, a current extraction factor is determined according to the current pulse recurrence frequency and a preset reference pulse recurrence frequency.

The preset reference pulse recurrence frequency may be adaptively set according to an actual demand, for example, may be set to be 2.6 kHz, 3.8 kHz, etc., which will not be specifically limited in present disclosure.

In some embodiments, the current extraction factor may be determined according to a ratio of the current pulse recurrence frequency to the preset reference pulse recurrence frequency.

In an implementation, the current extraction factor may be determined according to the following formula (1):

$$factor = \frac{{f}_{1}}{{f}_{2}} , \qquad ( 1 )$$

where factor represents the current extraction factor, f₁ represents the current pulse recurrence frequency, and f₂ represents the preset reference pulse recurrence frequency.

For example, if the current pulse recurrence frequency is 6, the preset reference pulse recurrence frequency is 3, the current extraction factor may be calculated to be 2 according to the formula (1).

At block 103, a target gradation sequence is extracted from a preset gradation set according to the current extraction factor.

The preset gradation set may be such a gradation set that is generated based on three primary colors, i.e., red (R), green (G) and blue (B), and has any data volume, such as 1024 gradations, 2048 gradations and so on, which will not be specifically limited in present disclosure. In an implementation, as shown in FIG. 2, the preset gradation set includes 1024 gradations.

Specifically, in some embodiments, after the current extraction factor is determined, a corresponding gradation sequence may be extracted from the preset gradation set according to the current extraction factor and taken as the target gradation sequence.

For example, if the preset gradation set includes 1024 gradations, and the current extraction factor is 2, extraction is performed every other gradation from the preset gradation set to obtain a gradation sequence having 512 gradations as the target gradation sequence.

It should be illustrated that, implementations of extracting the target gradation sequence from the preset gradation set according to the current extraction factor will be described in detail below and will not be elaborated here.

At block 104, a blood flow velocity currently detected by the ultrasonic system is displayed using a spectrum of the target gradation sequence.

In order to completely and effectively display the blood flow velocity currently detected, after the target gradation sequence is obtained, a corresponding spectrum may be constituted by the target gradation sequence, and the blood flow velocity currently detected is displayed using the spectrum.

It should be illustrated that, the blood flow velocity is a part of blood flow information, which is detected by a sensor of the ultrasonic system. Therefore, in some embodiments of the present disclosure, the blood flow displaying method further includes receiving the blood flow information from a sensor of the ultrasonic system. According to the blood flow information, the blood flow velocity currently detected by the ultrasonic system is displayed using the spectrum of the target gradation sequence.

Specifically, when blood flows in a blood vessel, the blood flow velocity is proportional to a blood flow volume and inversely proportional to a cross section of the blood vessel. For example, in a quiet state, an average blood flow velocity is about 18 to 22 cm/sec in aorta and about 0.3 to 0.7 in capillary. Therefore, in order to accurately and reliably present the blood flow velocity in the spectrum, in some embodiments, a blood flow velocity range corresponding to the current pulse recurrence frequency may be determined, and then one-to-one mapped to the spectrum of the target gradation sequence successively.

In some embodiments, a mapping mode of the blood flow velocity to the spectrum may be linear mapping. For example, if the blood flow velocity range outputted is in a range from −128 to +127, the blood flow velocities are mapped to color data of the spectrum point by point, i.e., the −128 of the blood flow velocity is mapped to a color represented by a negative maximum value, and the +127 of the blood flow velocity is mapped to a color represented by a positive maximum value.

In an implementation, the blood flow velocity range corresponding to the current pulse recurrence frequency may be determined according to formula (2), such as:

$$V = \frac{C ⋆ prf}{4 ⋆ {f}_{0}} , \qquad ( 2 )$$

where V represents the blood flow velocity detected under the current pulse recurrence frequency, C represents a ultrasound propagation velocity of the ultrasonic system, generally being 1540 m/s, prf represents the current pulse recurrence frequency, and f₀ represents a central frequency.

Further, in addition to the blood flow velocity, blood flow information actually detected includes a blood flow direction. Therefore, in embodiments of the present disclosure, the blood flow direction can be displayed together with the blood flow velocity currently detected by the ultrasonic system. It should be illustrated that, in the present disclosure, the blood flow information including the blood flow velocity and blood flow direction is acquired from a subject by a sensor of the ultrasonic system.

For example, if the blood flow velocity currently detected includes a low blood flow velocity and a high blood flow velocity, the blood flow direction includes a forward blood flow direction and a reversed blood flow direction, and the obtained target gradation sequence includes 1024 gradations, luminous yellow and dark red in 512 positive gradations of the target gradation sequence may be used to represent a highest-velocity forward blood flow and a lowest-velocity forward blood flow, respectively, and bright green and dark blue in 512 negative gradations of the target gradation sequence may be used to represent a highest-velocity reversed blood flow and a lowest-velocity reversed blood flow, respectively.

Accordingly, color information presented in the spectrum includes in turn: luminous yellow, transition colors (509 gradations) between the luminous yellow and the dark red, dark red, black (2 gradations), dark blue, transition colors (509 gradations) between the dark blue and the bright green, and bright green.

With the blood flow displaying method for an ultrasonic system provided in embodiments of the present disclosure, by determining the current pulse recurrence frequency of the ultrasonic system, determining the current extraction factor according to the current pulse recurrence frequency and the preset reference pulse recurrence frequency, extracting the target gradation sequence from the preset gradation set according to the current extraction factor, and displaying the blood flow velocity currently detected by the ultrasonic system using the spectrum of the target gradation sequence, the target gradation sequence is obtained, and the blood flow is displayed using the spectrum of the target gradation sequence. Therefore, the blood flow display is not only enhanced in gradation, but also may be optimized according to requirements of medical staffs, which greatly improves the flexibility of the blood flow display, and provides reliable basis and conditions for accurate diagnosis and analysis of the medical staffs.

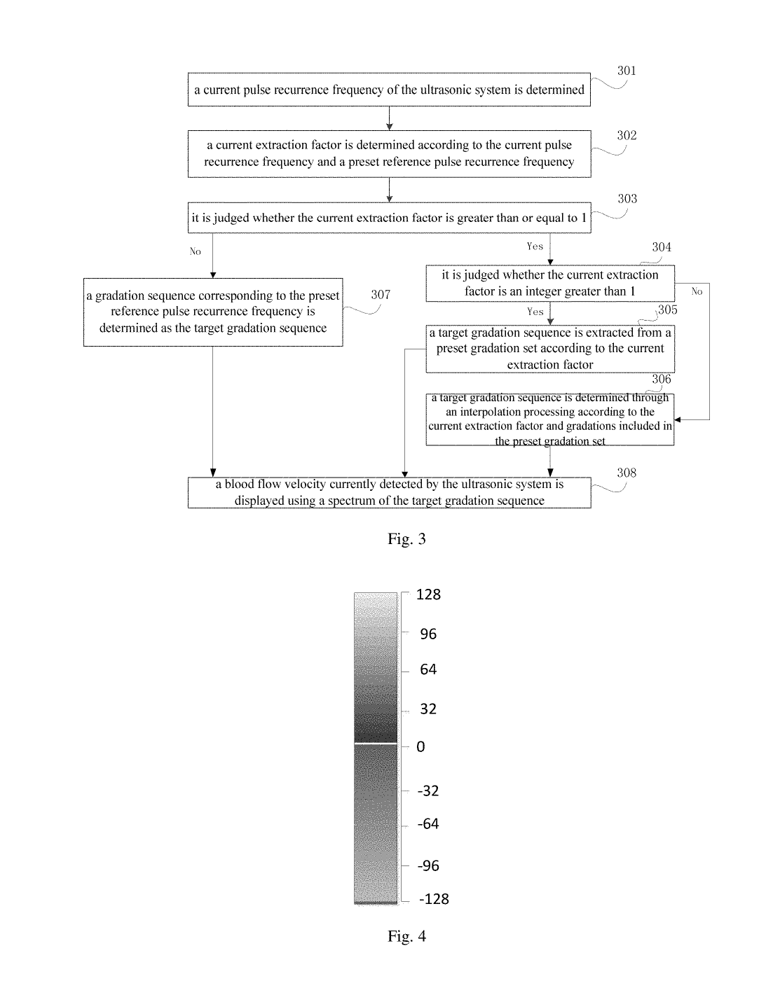

According to the above analysis, in embodiments of the present disclosure, the current extraction factor is determined according to the current pulse recurrence frequency and the preset reference pulse recurrence frequency, the target gradation sequence is extracted according to the current extraction factor, and the blood flow velocity currently detected by the ultrasonic system is displayed using the spectrum of the target gradation sequence. In implementations, as the current extraction factor determined according to the current pulse recurrence frequency and the preset reference pulse recurrence frequency may be an integer or an rational number, in order to accurately and effectively extract the target gradation sequence from the preset gradation set according to current extraction factor, different ways may be used to extract the target gradation sequence depending on specific situations of the extraction factor. Referring to FIG. 3, the above situations of the blood flow displaying method for an ultrasonic system of the present disclosure will be described in detail below.

FIG. 3 is a flow chart of a blood flow displaying method for an ultrasonic system according to embodiments of the present disclosure.

As shown in FIG. 3, the blood flow displaying method for an ultrasonic system according to embodiments of the present disclosure may include the following operations at blocks illustrated in FIG. 3.

At block 301, a current pulse recurrence frequency of the ultrasonic system is determined.

At block 302, a current extraction factor is determined according to the current pulse recurrence frequency and a preset reference pulse recurrence frequency.

At block 303, it is judged whether the current extraction factor is greater than or equal to 1, if yes, an operation at block 304 is executed, otherwise, an operation at block 307 is executed.

Specifically, by judging whether the current extraction factor is greater than or equal to 1, a way to obtain a target gradation sequence is determined. For example, if the extraction factor is greater than or equal to 1, the target gradation sequence is extracted from a preset gradation set directly according to the extraction factor.

At block 304, it is judged whether the current extraction factor is an integer greater than 1, if yes, an operation at block 305 is executed, otherwise, an operation at block 306 is executed.

At block 305, a target gradation sequence is extracted from a preset gradation set according to the current extraction factor.

In implementations, the target gradation sequence may be extracted from the preset gradation set through several ways, some of which will be illustrated below as examples.

As an implementation, the target gradation sequence is extracted with the same intervals from the preset gradation set according to the current extraction factor.

For example, if the extraction factor is 2, and the preset gradation set includes 1024 gradations, extraction is performed from the preset gradation set with 2 as the extraction factor, to obtain the target gradation sequence having 512 gradations.

As another implementation, extraction is respectively performed starting from a central position to each of two ends of the preset gradation set, according to the extraction factor, so as to obtain the target gradation sequence.

For example, if the extraction factor is 2, and the preset gradation set includes 1024 gradations, extraction is respectively performed starting from the central position to each of two ends of the preset gradation set with 2 as the extraction factor, to obtain the target gradation sequence having 512 gradations.

It should be illustrated that, the above implementations are only illustrative, and shall not be construed to limit the present disclosure.

At block 306, a target gradation sequence is determined through an interpolation processing according to the current extraction factor and gradations included in the preset gradation set.

That is, if it is judged that the current extraction factor k is a non-integer greater than 1, and k=a/b where both a and b are positive integers, the target gradation sequence may be determined through the interpolation processing.

For example, if

$$k = \frac{6}{5} ,$$

it can be determined that k is 1.2, the interpolation processing may be performed on 1.2 by 10 times, then based on an extraction factor obtained thereafter, extraction is performed every other 11 gradations from the preset gradation set having 1024 gradations to obtain a gradation sequence as the target gradation sequence.

In embodiments of the present disclosure, b gradations may be extracted per a gradations of the preset gradation set to constitute the target gradation sequence.

For example, if

$$k = \frac{6}{5} ,$$

5 gradations may be extracted per 6 gradations of the preset gradation set to constitute the target gradation sequence.

In embodiments of the present disclosure, the rule of extracting b gradations per a gradations of the preset gradation set may be extracting b gradations from a front part, a middle part, or a rear part of a gradations, which will not be limited herein.

At block 307, a gradation sequence corresponding to the preset reference pulse recurrence frequency is determined as the target gradation sequence.

The gradation sequence corresponding to the preset reference pulse recurrence frequency may be adaptively set according to an actual demand, and for example, as shown in FIG. 4, may be a gradation sequence having 256 gradations, which will not be limited herein.

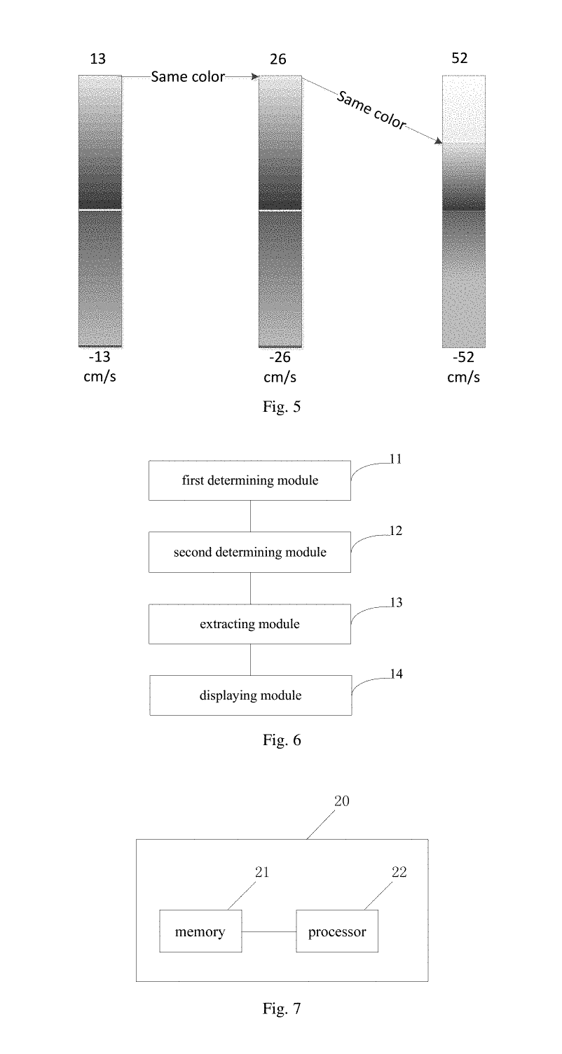

Specifically, when the extraction factor is less than 1, gradations extracted from the preset gradation set is not stretched, thereby decreasing a dynamic range of the blood flow display. Therefore, when the extraction factor is calculated to be less than 1, extraction operation of the target gradation sequence will not be performed, but directly taking the gradation sequence corresponding to the preset reference pulse recurrence frequency as the target gradation sequence to display blood flow information, such as the blood flow velocity, as shown in FIG. 5.

Commonly, in practice, in order to better display the blood flow situation, the number (M) of gradations in the preset gradation set is generally set to be greater than the number (N) of gradations displayed by the ultrasonic system, i.e., N is less than M.

Therefore, in some embodiments, before block 307, N gradations are extracted from the M gradations according to a preset rule to constitute the gradation sequence corresponding to the preset reference pulse recurrence frequency.

In an implementation, the gradation sequence corresponding to the preset reference pulse recurrence frequency may be obtained according to the following rules, which will be illustrated with reference to examples.

N/2 gradations are extracted starting from a central position to each of two ends of the M gradations, respectively.

For example, if M is 1024, and N is 256, 128 gradations may be extracted starting from gradation 0 towards each of positive and negative directions of the 1024 gradations, respectively, and 128 positive gradations and 128 negative gradations extracted thereby constitute the gradation sequence.

It should be illustrated that, in addition to the above implementations, other implementations may also be included, which will not be limited herein.

At block 308, a blood flow velocity currently detected by the ultrasonic system is displayed using a spectrum of the target gradation sequence.

With the blood flow displaying method for an ultrasonic system provided in embodiments of the present disclosure, after the current extraction factor is determined, an extraction way corresponding to the current extraction factor is obtained by judging the current extraction factor, the target gradation sequence is extracted from the preset gradation set according to the current extraction factor or the gradation sequence corresponding to the preset reference pulse recurrence frequency is directly determined as the target gradation sequence, and the blood flow velocity currently detected by the ultrasonic system is displayed using the spectrum of the obtained target gradation sequence. Therefore, the corresponding way to obtain the target gradation sequence is obtained by judging the extraction factor, by which a corresponding target gradation sequence is obtained, such that the display of the dynamic range of the detected blood flow velocities meets the actual demands more and an effect of the blood flow display is improved.

In the following, the blood flow displaying apparatus for an ultrasonic system according to embodiments of the present disclosure will be described with reference to drawings.

FIG. 6 is a schematic block diagram of a blood flow displaying apparatus for an ultrasonic system according to embodiments of the present disclosure.

As shown in FIG. 6, the blood flow displaying apparatus includes: a first determining module 11, a second determining module 12, an extracting module 13, and a displaying module 14.

The first determining module 11 is configured to determine a current pulse recurrence frequency of the ultrasonic system.

The second determining module 12 is configured to determine a current extraction factor according to the current pulse recurrence frequency and a preset reference pulse recurrence frequency.

The extracting module 13 is configured to extract a target gradation sequence from a preset gradation set according to the current extraction factor.

The displaying module 14 is configured to display a blood flow velocity currently detected by the ultrasonic system using a spectrum of the target gradation sequence.

In some embodiments of the present disclosure, the blood flow displaying apparatus further includes a receiving module 15 configured to receive blood flow information from a sensor of the ultrasonic system.

It should be illustrated that, regarding implementations and technical principles of the blood flow displaying apparatus for an ultrasonic system, reference can be made to explanations and illustrations of the blood flow displaying method for an ultrasonic system as described hereinbefore, which will not be elaborated herein.

With the blood flow displaying apparatus for an ultrasonic system provided in embodiments of the present disclosure, by determining the current pulse recurrence frequency of the ultrasonic system, determining the current extraction factor according to the current pulse recurrence frequency and the preset reference pulse recurrence frequency, extracting the target gradation sequence from the preset gradation set according to the current extraction factor, and displaying the blood flow velocity currently detected by the ultrasonic system using the spectrum of the target gradation sequence, the target gradation sequence is obtained, and the blood flow is displayed using the spectrum of the target gradation sequence. Therefore, the blood flow display is not only enhanced in gradation, but also may be optimized according to requirements of medical staffs, which greatly improves the flexibility of the blood flow display, and provides reliable basis and conditions for accurate diagnosis and analysis of the medical staffs.

In embodiments of the present disclosure, a computer device is provided.

FIG. 7 is a schematic diagram of a computer device according to embodiments of the present disclosure.

As shown in FIG. 7, the computer device 20 includes: a processor 22; and a memory 21 having stored therein computer programs executable by the processor 22, when executed by the processor 22, the computer programs cause the processor 22 to implement the blood flow displaying method for an ultrasonic system as described hereinbefore. The blood flow displaying method for an ultrasonic system includes: determining a current pulse recurrence frequency of the ultrasonic system; determining a current extraction factor according to the current pulse recurrence frequency and a preset reference pulse recurrence frequency; extracting a target gradation sequence from a preset gradation set according to the current extraction factor; and displaying a blood flow velocity currently detected by the ultrasonic system using a spectrum of the target gradation sequence.

In some embodiments of the present disclosure, the blood flow displaying method further includes receiving blood flow information from a sensor of the ultrasonic system.

It should be illustrated that, regarding implementations and technical principles of the computer device, reference can be made to explanations and illustrations of the blood flow displaying method for an ultrasonic system as described hereinbefore, which will not be elaborated herein.

With the computer device provided in embodiments of the present disclosure, by determining the current pulse recurrence frequency of the ultrasonic system, determining the current extraction factor according to the current pulse recurrence frequency and the preset reference pulse recurrence frequency, extracting the target gradation sequence from the preset gradation set according to the current extraction factor, and displaying the blood flow velocity currently detected by the ultrasonic system using the spectrum of the target gradation sequence, the target gradation sequence is obtained, and the blood flow is displayed using the spectrum of the target gradation sequence. Therefore, the blood flow display is not only enhanced in gradation, but also may be optimized according to requirements of medical staffs, which greatly improves the flexibility of the blood flow display, and provides reliable basis and conditions for accurate diagnosis and analysis of the medical staffs.

In embodiments of the present disclosure, a computer-readable storage medium is provided.

The computer-readable storage medium has stored therein computer programs that, when executed by a processor, causes the processor to implement the blood flow displaying method for an ultrasonic system as described hereinbefore. The blood flow displaying method for an ultrasonic system includes: determining a current pulse recurrence frequency of the ultrasonic system; determining a current extraction factor according to the current pulse recurrence frequency and a preset reference pulse recurrence frequency; extracting a target gradation sequence from a preset gradation set according to the current extraction factor; and displaying a blood flow velocity currently detected by the ultrasonic system using a spectrum of the target gradation sequence.

In embodiments of the present disclosure, there is provided a blood flow displaying device for an ultrasonic system. The blood flow displaying device is configured to execute the blood flow displaying method for an ultrasonic system as described hereinbefore.

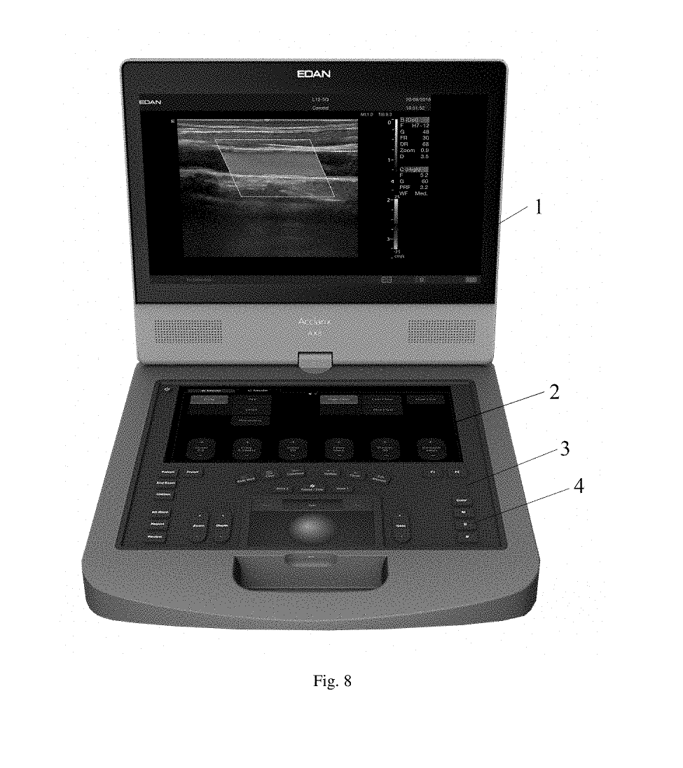

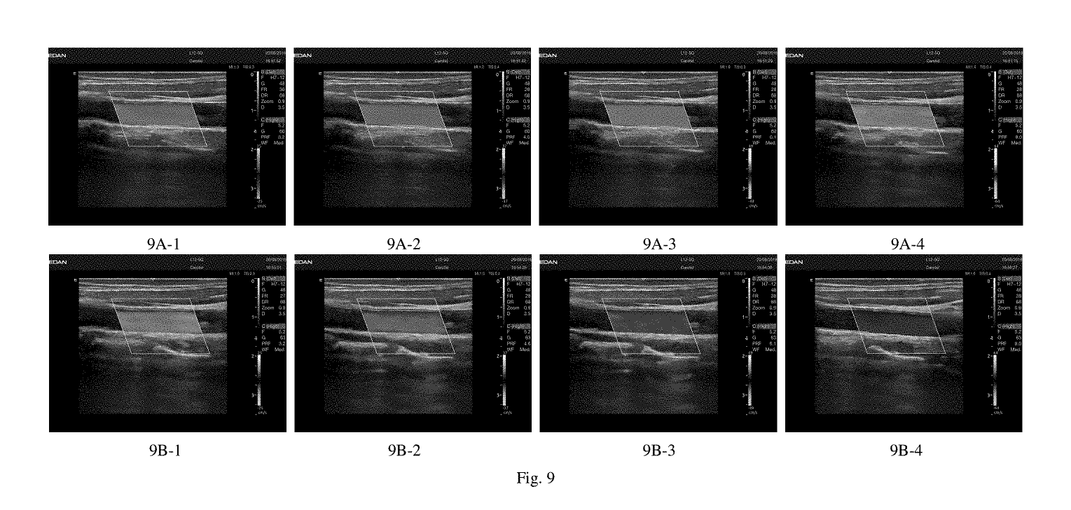

FIG. 8 is a schematic diagram of the blood flow displaying device according to embodiments of the present disclosure, as shown in FIG. 8. The flow displaying device includes an upper display screen 1; a lower display screen 2, an operation panel 3; a processor; and a memory having stored therein computer programs executable by the processor. The operation panel 3 includes a plurality of buttons 4 for operating the blood flow displaying device. The lower display screen 2 includes a plurality of operating modes, among which an operating mode can be chose by a user as required. When the operating mode such as for displaying blood flow is determined, the processor is activated, and the computer programs cause the processor to implement the blood flow displaying method for an ultrasonic system as described hereinbefore. The display screen 1 is configured to display a result obtained by the blood flow displaying method of the present disclosure. In embodiments of the present disclosure, the display screen 1 is configured to display a blood flow velocity currently detected by the ultrasonic system using a spectrum of the target gradation sequence, as shown in diagrams 9A-1 to 9A-4 of FIG. 9.

It should be illustrated that, regarding implementations and technical principles of the blood flow displaying device for an ultrasonic system, reference can be made to explanations and illustrations of the blood flow displaying method for an ultrasonic system as described hereinbefore, which will not be elaborated herein.

It should be illustrated that, as described above, the blood flow velocity is determined according to blood flow information, which is detected by a sensor of the ultrasonic system. Therefore, in some embodiments of the present disclosure, the blood flow displaying device is further configured to receive blood flow information from a sensor of the ultrasonic system. According to the blood flow information, the blood flow velocity is displayed by the display screen 1 of the blood flow displaying device using the spectrum of the target gradation sequence.

FIG. 9 illustrates comparisons between blood flow displaying results obtained by the blood flow displaying method according to embodiments of the present disclosure and that obtained by a method in related art, where diagrams 9A-1 to 9A-4 illustrate blood flow display results and corresponding spectrums obtained using the blood flow displaying method according to embodiments of the present disclosure; and diagrams 9B-1 to 9B-4 illustrate blood flow displaying results and corresponding spectrums obtained using the blood flow displaying method in the related art, in which the prf increases gradually from 9A-1 to 9A-4 and from 9B-1 to 9B-4, and the diagrams 9A-1 and 9B-1 are obtained at the same prf, and this is also true for diagrams 9A-2 and 9B-2, for diagrams 9A-3 and 9B-3, and for diagrams 9A-4 and 9B-4. It should be illustrated that these diagrams are obtained using the blood flow displaying device as illustrated in FIG. 8.

From comparisons of these diagrams, it can be seen that the blood flow displayed with the method according to embodiments of the present disclosure looks bright no matter the prf is; however, the blood flow displayed with the method in the related art darkens gradually and imaging of blood flow is not distinct enough with the increase of the prf. Moreover, the blood flow displayed with the method according to embodiments of the present disclosure looks brighter than that displayed with the method in the related art at the same prf.

Reference throughout this specification to “an embodiment,” “some embodiments,” “one embodiment”, “another example,” “an example,” “a specific example,” or “some examples,” means that a particular feature, structure, material, or characteristic described in connection with the embodiment or example is included in at least one embodiment or example of the present disclosure.

In addition, terms such as “first” and “second” are used herein for purposes of description and are not intended to indicate or imply relative importance or significance or to imply the number of indicated technical features. Thus, the feature defined with “first” and “second” may comprise one or more of this feature.

Any process or method described in a flow chart or described herein in other ways may be understood to include one or more modules, segments or portions of codes of executable instructions for achieving specific logical functions or steps in the process, and the scope of a preferred embodiment of the present disclosure includes other implementations, in which the order of execution is different from what is shown or discussed, including executing functions in a substantially simultaneous manner or in an opposite order according to the related functions. These and other aspects should be understood by those skilled in the art.

It should be understood that each part of the present disclosure may be realized by the hardware, software, firmware or their combination. In the above embodiments, a plurality of steps or methods may be realized by the software or firmware stored in the memory and executed by the appropriate instruction execution system. For example, if it is realized by the hardware, likewise in another embodiment, the steps or methods may be realized by one or a combination of the following techniques known in the art: a discrete logic circuit having a logic gate circuit for realizing a logic function of a data signal, an application-specific integrated circuit having an appropriate combination logic gate circuit, a programmable gate array (PGA), a field programmable gate array (FPGA), etc.

It can be understood that all or part of the steps in the method of the above embodiments can be implemented by instructing related hardware via programs, the program may be stored in a computer readable storage medium, and the program includes one step or combinations of the steps of the method when the program is executed.

The computer readable storage medium may be, but is not limited to, read-only memories, magnetic disks, or optical disks.

Although embodiments have been shown and described, it would be appreciated by those skilled in the art that the above embodiments cannot be construed to limit the present disclosure, and changes, alternatives, and modifications can be made in the embodiments without departing from spirit, principles and scope of the present disclosure.

What is claimed is:

1. A blood flow displaying method for an ultrasonic system, comprising:
determining a current pulse recurrence frequency of the ultrasonic system;
determining a current extraction factor according to the current pulse recurrence frequency and a preset reference pulse recurrence frequency;
extracting a target gradation sequence from a preset gradation set according to the current extraction factor; and
displaying a blood flow velocity currently detected by the ultrasonic system using a spectrum of the target gradation sequence.

2. The method according to claim 1, wherein determining a current extraction factor comprises: determining the current extraction factor according to a ratio of the current pulse recurrence frequency to the preset reference pulse recurrence frequency.

3. The method according to claim 1, wherein extracting a target gradation sequence from a preset gradation set comprises:
extracting the target gradation sequence from the preset gradation set according to the current extraction factor if the current extraction factor is greater than or equal to 1; or
determining a gradation sequence corresponding to the preset reference pulse recurrence frequency as the target gradation sequence if the current extraction factor is less than 1.

4. The method according to claim 3, wherein the preset gradation set comprises M gradations, and the number of gradations displayed by the ultrasonic system is N, and N is less than M;
wherein before determining the gradation sequence corresponding to the preset reference pulse recurrence frequency as the target gradation sequence, the method further comprises:
extracting N gradations from the M gradations according to a preset rule to constitute the gradation sequence corresponding to the preset reference pulse recurrence frequency.

5. The method according to claim 4, wherein extracting N gradations from the M gradations according to a preset rule comprises: extracting N/2 gradations starting from a central position to each of two ends of the M gradations, respectively.

6. The method according to claim 3, wherein if the current extraction factor k is a non-integer greater than 1, and k=a/b where both a and b are positive integers, extracting the target gradation sequence from the preset gradation set according to the current extraction factor comprises:
determining the target gradation sequence through an interpolation processing according to the current extraction factor and gradations comprised in the preset gradation set; or
extracting b gradations per a gradations of the preset gradation set to constitute the target gradation sequence.

7. The method according to claim 2, wherein extracting a target gradation sequence from a preset gradation set comprises:
extracting the target gradation sequence from the preset gradation set according to the current extraction factor if the current extraction factor is greater than or equal to 1; or
determining a gradation sequence corresponding to the preset reference pulse recurrence frequency as the target gradation sequence if the current extraction factor is less than 1.

8. The method according to claim 7, wherein the preset gradation set comprises M gradations, and the number of gradations displayed by the ultrasonic system is N, and N is less than M;
wherein before determining the gradation sequence corresponding to the preset reference pulse recurrence frequency as the target gradation sequence, the method further comprises:
extracting N gradations from the M gradations according to a preset rule to constitute the gradation sequence corresponding to the preset reference pulse recurrence frequency.

9. The method according to claim 8, wherein extracting N gradations from the M gradations according to a preset rule comprises: extracting N/2 gradations starting from a central position to each of two ends of the M gradations, respectively.

10. The method according to claim 7, wherein if the current extraction factor k is a non-integer greater than 1, and k=a/b where both a and b are positive integers, extracting the target gradation sequence from the preset gradation set according to the current extraction factor comprises:
determining the target gradation sequence through an interpolation processing according to the current extraction factor and gradations comprised in the preset gradation set; or
extracting b gradations per a gradations of the preset gradation set to constitute the target gradation sequence.

11. The method according to claim 1, wherein displaying a blood flow velocity currently detected by the ultrasonic system using a spectrum of the target gradation sequence comprises:
determining a blood flow velocity range corresponding to the current pulse recurrence frequency; and
one-to-one mapping the blood flow velocity range to the spectrum of the target gradation sequence successively.

12. A computer device, comprising:
a processor; and
a memory having stored therein computer programs executable by the processor,
wherein when executed by the processor, the computer programs cause the processor to implement a blood flow displaying method for an ultrasonic system, the method comprising:
determining a current pulse recurrence frequency of the ultrasonic system;
determining a current extraction factor according to the current pulse recurrence frequency and a preset reference pulse recurrence frequency;
extracting a target gradation sequence from a preset gradation set according to the current extraction factor; and
displaying a blood flow velocity currently detected by the ultrasonic system using a spectrum of the target gradation sequence.

13. The computer device according to claim 12, wherein determining a current extraction factor comprises: determining the current extraction factor according to a ratio of the current pulse recurrence frequency to the preset reference pulse recurrence frequency.

14. The computer device according to claim 12, wherein extracting a target gradation sequence from a preset gradation set comprises:
extracting the target gradation sequence from the preset gradation set according to the current extraction factor if the current extraction factor is greater than or equal to 1; or
determining a gradation sequence corresponding to the preset reference pulse recurrence frequency as the target gradation sequence if the current extraction factor is less than 1.

15. The computer device according to claim 14, wherein the preset gradation set comprises M gradations, and the number of gradations displayed by the ultrasonic system is N, and N is less than M;
wherein before determining the gradation sequence corresponding to the preset reference pulse recurrence frequency as the target gradation sequence, the method further comprises:
extracting N gradations from the M gradations according to a preset rule to constitute the gradation sequence corresponding to the preset reference pulse recurrence frequency.

16. The computer device according to claim 15, wherein extracting N gradations from the M gradations according to a preset rule comprises: extracting N/2 gradations starting from a central position to each of two ends of the M gradations, respectively.

17. The computer device according to claim 14, wherein if the current extraction factor k is a non-integer greater than 1, and k=a/b where both a and b are positive integers, extracting the target gradation sequence from the preset gradation set according to the current extraction factor comprises:
determining the target gradation sequence through an interpolation processing according to the current extraction factor and gradations comprised in the preset gradation set; or
extracting b gradations per a gradations of the preset gradation set to constitute the target gradation sequence.

18. The computer device according to claim 12, wherein displaying a blood flow velocity currently detected by the ultrasonic system using a spectrum of the target gradation sequence comprises:
determining a blood flow velocity range corresponding to the current pulse recurrence frequency; and
one-to-one mapping the blood flow velocity range to the spectrum of the target gradation sequence successively.

19. A computer-readable storage medium having stored therein computer programs that, when executed by a processor, causes the processor to implement a blood flow displaying method for an ultrasonic system, the method comprising:
determining a current pulse recurrence frequency of the ultrasonic system;
determining a current extraction factor according to the current pulse recurrence frequency and a preset reference pulse recurrence frequency;
extracting a target gradation sequence from a preset gradation set according to the current extraction factor; and
displaying a blood flow velocity currently detected by the ultrasonic system using a spectrum of the target gradation sequence.

20. The method according to claim 1, further comprising: receiving blood flow information from a sensor of the ultrasonic system.
